(19) United States
(12) Patent Application Publication
(10) Pub. No.: US 2003/0049265 A1
(43) Pub. Date: Mar. 13, 2003

(54) Heliobacter pylori antigen

(76) Inventors: Margaret Dunkley, Elemore Vale (AU); Simon Harris, Canungra (AU)

Correspondence Address: STERNE, KESSLER, GOLDSTEIN & FOX PLLC, 1100 NEW YORK AVENUE, N.W., SUITE 600, WASHINGTON, DC, 20005-3934, US

(21) Appl. No.: 09/855,698

(22) Filed: May 16, 2001

Related U.S. Application Data
(63) Continuation of application No. PCT/GB99/03759, filed on Nov. 11, 1999.

(30) Foreign Application Priority Data
Nov. 17, 1998 (GB) 9825184.6

(51) Int. Cl. G01N 33/53; A61K 39/00; A61K 39/38; A61K 39/02; C07K 1/00; C07K 14/00; C07K 17/00

(52) U.S. Cl. 424/184.1; 530/350; 435/7.1

(57) ABSTRACT

A novel antigen derived from H.pyloria is disclosed. Its diagnostic and therapeutic use is also described, as are kits comprising the antigen and/or nucleic acid molecules coding for the antigen.

This is a continuation of International Application No. PCT/GB99/03759, with an International Filing Date of Nov. 11, 1999, the content of which is fully incorporated by reference.

The present invention relates to an antigen derived from H.pylori. The use of this antigen as an immunogen, together with pharmaceutical compositions comprising it, particularly vaccines, are also provided as are recombinant nucleic acid molecules encoding the antigen, vectors incorporating such nucleic acid molecules and host cells carrying such vectors.

H.pylori is a gram negative bacteria that has been strongly implicated in chronic active gastritis and peptic ulcer disease (Marshall et al, Medica Journal of Australia, 142:439-444 (1985); Buck, G. E., Journal of Clinical Microbiology, 3:1-12 (1990)). The original focus of research in the area of H.pylori antigens was for the purpose of diagnosis. However, interest was also centred on the need to provide a vaccine which would be effective against this common organism. A number of patent filings have disclosed candidate antigens for such vaccines, including WO96/25430, WO98/32768 and UK patent application No. 9806039.5.

However, there is a continuing need to provide further antigens to ensure that any vaccine has the fullest possible effectiveness, specificity and protection across strains. We have now isolated and identified an antigen which demonstrates good protective properties against H.pylori infection.

Thus, in a first aspect the present invention provide an H.pylori antigenic protein, having a molecular weight of 35 kDa, as measured by SDS-PAGE under reducing or non-reducing conditions and having the amino acid sequence:

MAKEILVAYGVDIDAVAGWLGSYGGEDSPDDISRGLFAGEVGIPRLLKLFKKY HLPATWFSPGHSIETFSEQMKMIVDAGHEVGAHGYSHENPIAMTAKQEEDVL LKSVELIKDLTGKAPTGYVAPWWEFSNITNELLLKHGFKYDHSLMHNDFTPY YVRVGDSWSKIDYSLEAKDWMKPLIRGVETDLVEIPANWYLDDLPPMMFIKK SPNSFGFVSPHDIGQMWIDQFDWVYREMDYAVFSMTIHPDVSARPQVLLMHE KIIEHINKHEGVRWVTFNEIADDFLKRNPRKK.

The protein of the present invention may be provided in substantially pure form. For example, it may be provided in a form which is substantially free of other proteins.

As discussed herein, the protein of the invention is useful as antigenic material. Such material can be “antigenic” and/or “immunogenic”. Generally, “antigenic” is taken to mean that the protein is capable of being used to raise antibodies or indeed is capable of inducing an antibody response in a subject. “Immunogenic” is taken to mean that the protein is capable of eliciting a protective immune response in a subject. Thus, in the latter case, the protein may be capable of not only generating an antibody response but, in addition, a non-antibody based immune response.

The skilled person will appreciate that homologues or derivatives of the protein of the invention will also find use in the context of the present invention, ie as antigenic/immunogenic material. Thus, for instance proteins which include one or more additions, deletions, substitutions or the like are encompassed by the present invention. In addition, it may be possible to replace one amino acid with another of similar “type”. For instance replacing one hydrophobic amino acid with another.

One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of identity analysis are contemplated in the present invention.

In the case of homologues and derivatives, the degree of identity with the protein described herein is less important than that the homologue or derivative should retain the antigenicity or immunogenicity of the original protein. However, suitably, homologues or derivatives having at least 60% similarity (as discussed above) with the proteins or polypeptides described herein are provided. Preferably, homologues or derivatives having at least 70% similarity, more preferably at least 80% similarity are provided. Most preferably, homologues or derivatives having at least 90% or even 95% similarity are provided.

In an alternative approach, the homologues or derivatives could be fusion proteins, incorporating moieties which render purification easier, for example by effectively tagging the desired protein or polypeptide. It may be necessary to remove the “tag” or it may be the case that the fusion protein itself retains sufficient antigenicity to be useful.

In an additional aspect of the invention there are provided antigenic/immunogenic fragments of the protein of the invention, or of homologues or derivatives thereof.

For fragments of the proteins or polypeptides described herein, or of homologues or derivatives thereof, the situation is slightly different. It is well known that is possible to screen an antigenic protein or polypeptide to identify epitopic regions, ie those regions which are responsible for the protein or polypeptide's antigenicity or immunogenicity. Methods for carrying out such screening are well known in the art. Thus, the fragments of the present invention should include one or more such epitopic regions or be sufficiently similar to such regions to retain their antigenic/immunogenic properties. Thus, for fragments according to the present invention the degree of identity is perhaps irrelevant, since they may be 100% identical to a particular part of a protein or polypeptide, homologue or derivative as described herein. The key issue, once again, is that the fragment retains the antigenic/immunogenic properties.

Thus, what is important for homologues, derivatives and fragments is that they possess at least a degree of the antigenicity/immunogenicity of the protein or polypeptide from which they are derived.

The N-terminal sequence of the protein of the invention was used to screen the TIGR database. A match was found, designated as HP0310. The function of the protein was (and indeed still is) unknown and no information concerning its antigenicity/immunogenicity was of course provided by the database.

Gene cloning techniques may be used to provide the protein of the invention in substantially pure form. These techniques are disclosed, for example, in J. Sambrook et al Molecular Cloning 2nd Edition, Cold Spring Harbor Laboratory Press (1989). Thus, in a third aspect, the present invention provides a recombinant nucleic acid molecule comprising or consisting of:

(i) the sequence:

ATGGCAAAAGAAATTTTAGTGGCTTATGGTGTGGATATTGATGCGGTGGC
TGGTTGGTTAGGGAGCTATGGTGGGGAGGATTCGCCTGATGATATTTCGC
GCGGGCTTTTTGCGGGTGAAGTGGGGATCCCACGGCTTTTGAAATTGTTT
AAAAAATACCATCTCCCGGCGACTTGGTTTTCGCCGGGGCATTCTATTGA
AACTTTCTCTGAACAAATGAAAATGATCGTGGATGCAGGGCATGAAGTGG
GCGCGCATGGGTATTCGCATGAAAACCCTATCGCTATGACGGCCAAGCAA
GAAGAAGACGTTTTGTTAAAAAGCGTTGAGTTGATTAAAGATCTCACCGG
CAAAGCCCCCACAGGCTATGTGGCGCCGTGGTGGGAGTTTTCTAATATCA
CTAATGAATTGCTTTTAAAACACGGCTTCAAATACGACCACTCGCTCATG
CACAATGATTTCACGCCCTATTATGTGCGCGTGGGGGATAGTTGGAGCAA
GATTGATTATAGTTTGGAAGCTAAGGATTGGATGAAGCCTTTAATCCGTG
GGGTGGAAACCGATCTGGTGGAAATCCCTGCGAACTGGTATTTGGACGAT
TTACCGCCGATGATGTTCATCAAAAAGTCCCCCAATAGTTTTGGTTTTGT
AAGTCCGCACGATATAGGGCAAATGTGGATCGATCAATTTGATTGGGTTT
ATCGTGAGATGGATTATGCGGTGTTTAGCATGACAATCCACCCTGATGTG
AGCGCCCGTCCGCAAGTGTTGCTCATGCATGAAAAAATCATTGAGCATAT
CAACAAGCACGAGGGCGTGCGTTGGGTAACATTCAATGAAATCGCTGATG
ATTTCTTAAAACGAAACCCTAGAAAAAAA.;

(ii) a sequence which is complementary to the sequence in (i);

(iii) a sequence which codes for the same protein, as those sequences of (i) or (ii);

(iv) a sequence which has substantial identity with any of those of (i), (ii) and (iii);

(v) a sequence which codes for a homologue, derivative or fragment of the protein as described herein.

The nucleic acid molecules of the invention may include a plurality of such sequences, and/or fragments. The skilled person will appreciate that the present invention can include novel variants of those particular novel nucleic acid molecules which are exemplified herein. Such variants are encompassed by the present invention. These may occur in nature, for example because of strain variation. For example, additions, substitutions and/or deletions are included. In addition, and particularly when utilising microbial expression systems, one may wish to engineer the nucleic acid sequence by making use of known preferred codon usage in the particular organism being used for expression. Thus, synthetic or non-naturally occurring variants are also included within the scope of the invention.

When comparing nucleic acid sequences for the purposes of determining the degree of homology or identity one can use programs such as BESTFIT and GAP (both from the Wisconsin Genetics Computer Group (GCG) software package) BESTFIT, for example, compares two sequences and produces an optimal alignment of the most similar segments. GAP enables sequences to be aligned along their whole length and finds the optimal alignment by inserting spaces in either sequence as appropriate. Suitably, in the context of the present invention when discussing identity of nucleic acid sequences, the comparison is made by alignment of the sequences along their whole length.

Preferably, sequences which have substantial identity have at least 50% sequence identity, desirably at least 75% sequence identity and more desirably at least 90 or at least 95% sequence identity with said sequences. In some cases the sequence identity may be 99% or above.

Desirably, the term “substantial identity” indicates that said sequence has a greater degree of identity with the sequence described herein than with prior art nucleic acid sequences.

It should however be noted that the present invention includes within its scope all possible sequences coding for the novel gene product described herein, or a novel part thereof.

The nucleic acid molecule may be in isolated or recombinant form. It may be incorporated into a vector and the vector may be incorporated into a host. Such vectors and suitable hosts form yet further aspects of the present invention.

Therefore, for example, by using probes based upon the nucleic acid sequence provided herein, the gene in H.pylori can be identified. It can then be excised using restriction enzymes and cloned into a vector. The vector can be introduced into a suitable host for expression.

Nucleic acid molecules of the present invention may be obtained from H.pylori by the use of appropriate probes complementary to part of the sequences of the nucleic acid molecules. Restriction enzymes or sonication techniques can be used to obtain appropriately sized fragments for probing.

Alternatively PCR techniques may be used to amplify a desired nucleic acid sequence. Thus the sequence data provided herein can be used to design primers for use in PCR so that a desired sequence, including the whole gene or fragments thereof, can be targeted and then amplified to a high degree.

Typically primers will be at least 15-25 nucleotides long.

As a further alternative chemical synthesis may be used. This may be automated. Relatively short sequences may be chemically synthesised and ligated together to provide a longer sequence.

In yet a further aspect the present invention provides an immunogenic/antigenic composition comprising the protein of the invention, or a homologue or derivative thereof, and/or fragments of any of these. In preferred embodiments, the immunogenic/antigenic composition is a vaccine or is for use in a diagnostic assay.

In the case of vaccines suitable additional excipients, diluents, adjuvants or the like may be included. Numerous examples of these are well known in the art.

It is also possible to utilise the nucleic acid sequences described herein in the preparation of so-called DNA vaccines. Thus, the invention also provides a vaccine composition comprising one or more nucleic acid sequences as defined herein. DNA vaccines are described in the art (see for instance, Donnelly et al , Ann. Rev. Immunol., 15:617-648 (1997)) and the skilled person can use such art described techniques to produce and use DNA vaccines according to the present invention.

In addition, the protein described herein, its homologues or derivatives, and/or fragments of any of these, can be used in methods of detecting/diagnosing H.pylori. Such methods can be based on the detection of antibodies against such proteins which may be present in a subject. Therefore the present invention provides a method for the detection/diagnosis of H.pylori which comprises the step of bringing into contact a sample to be tested with the protein, or homologue, derivative or fragment thereof, as described herein. Suitably, the sample is a biological sample, such as a tissue sample or a sample of blood or saliva obtained from a subject to be tested.

In an alternative approach, the protein described herein, or homologues, derivatives and/or fragments thereof, can be used to raise antibodies, which in turn can be used to detect the antigens, and hence H.pylori. Such antibodies form another aspect of the invention. Antibodies within the scope of the present invention may be monoclonal or polyclonal.

Polyclonal antibodies can be raised by stimulating their production in a suitable animal host (e.g. a mouse, rat, guinea pig, rabbit, sheep, goat or monkey) when a protein as described herein, or a homologue, derivative or fragment thereof, is injected into the animal. If desired, an adjuvant may be administered together with the protein. Well-known adjuvants include Freund's adjuvant (complete and incomplete) and aluminium hydroxide. The antibodies can then be purified by virtue of their binding to a protein as described herein.

Monoclonal antibodies can be produced from hybridomas. These can be formed by fusing myeloma cells and spleen cells which produce the desired antibody in order to form an immortal cell line. Thus the well-known Kohler & Milstein technique (Nature 256 (1975)) or subsequent variations upon this technique can be used.

Techniques for producing monoclonal and polyclonal antibodies that bind to a particular polypeptide/protein are now well developed in the art. They are discussed in standard immunology textbooks, for example in Roitt et al, Immunology second edition (1989), Churchill Livingstone, London.

In addition to whole antibodies, the present invention includes derivatives thereof which are capable of binding to proteins etc as described herein. Thus the present invention includes antibody fragments and synthetic constructs. Examples of antibody fragments and synthetic constructs are given by Dougall et al in Tibtech 12 372-379 (September 1994).

Antibody fragments include, for example, Fab, F(ab′)₂ and Fv fragments. Fab fragments (These are discussed in Roitt et al [supra]). Fv fragments can be modified to produce a synthetic construct known as a single chain Fv (scFv) molecule. This includes a peptide linker covalently joining Vₕ and V_{I} regions, which contributes to the stability of the molecule. Other synthetic constructs that can be used include CDR peptides. These are synthetic peptides comprising antigen-binding determinants. Peptide mimetics may also be used. These molecules are usually conformationally restricted organic rings that mimic the structure of a CDR loop and that include antigen-interactive side chains.

Synthetic constructs include chimaeric molecules. Thus, for example, humanised (or primatised) antibodies or derivatives thereof are within the scope of the present invention. An example of a humanised antibody is an antibody having human framework regions, but rodent hypervariable regions. Ways of producing chimaeric antibodies are discussed for example by Morrison et al in PNAS, 81, 6851-6855 (1984) and by Takeda et al in Nature. 314, 452-454 (1985).

Synthetic constructs also include molecules comprising an additional moiety that provides the molecule with some desirable property in addition to antigen binding. For example the moiety may be a label (e.g. a fluorescent or radioactive label). Alternatively, it may be a pharmaceutically active agent.

Antibodies, or derivatives thereof, find use in detection/diagnosis of H.pylori. Thus, in another aspect the present invention provides a method for the detection/diagnosis of H.pylori which comprises the step of bringing into contact a sample to be tested and antibodies capable of binding to the protein described herein, or to homologues, derivatives and/or fragments thereof.

In addition, so-called “Affibodies” may be utilised. These are binding proteins selected from combinatorial libraries of an alpha-helical bacterial receptor domain (Nord et al, ) Thus, Small protein domains, capable of specific binding to different target proteins can be selected using combinatorial approaches.

It will also be clear that the nucleic acid sequences described herein may be used to detect/diagnose H.pylori. Thus, in yet a further aspect, the present invention provides a method for the detection/diagnosis of H.pylori which comprises the step of bringing into contact a sample to be tested with at least one nucleic acid sequence as described herein. Suitably, the sample is a biological sample, such as a tissue sample or a sample of blood or saliva obtained from a subject to be tested. Such samples may be pre-treated before being used in the methods of the invention. Thus, for example, a sample may be treated to extract DNA. Then, DNA probes based on the nucleic acid sequences described herein (ie usually fragments of such sequences) may be used to detect nucleic acid from .H.pylori.

In additional aspects, the present invention provides:

(a) a method of vaccinating a subject against H.pylori which comprises the step of administering to a subject the protein of the invention, or a derivative, homologue or one or more fragments thereof, or an immunogenic composition of the invention;

(b) a method of vaccinating a subject against H.pylori which comprises the step of administering to a subject a nucleic acid molecule as defined herein;

(c) a method for the prophylaxis or treatment of H.pylori infection which comprises the step of administering to a subject the protein of the invention, or a derivative, homologue or one or more fragments thereof, or an immunogenic composition of the invention;

(d) a method for the prophylaxis or treatment of H.pylori infection which comprises the step of administering to a subject a nucleic acid molecule as defined herein;

(e) a kit for use in detecting/diagnosing H.pylori infection comprising the protein of the invention, or a homologue, derivative or one or more fragments thereof, or an antigenic composition of the invention; and

(f) a kit for use in detecting/diagnosing H.pylori infection comprising one or more nucleic acid molecules as defined herein;

(g) a kit for use in detecting/diagnosing H.pylori infection comprising one or more antibodies as defined herein;

(h) the use of the protein of the invention, or a homologue, derivative or one or more fragments thereof, or an antigenic composition of the invention in the manufacture of a medicament for the prophylaxis or treatment of H.pylori infection;

(i) the use of one or more nucleic acid molecules as defined herein, or one or more fragments thereof in the manufacture of a medicament for the prophylaxis or treatment of H.pylori infection.

The invention will now be described with reference to the following examples, which should not be construed as in any way limiting the scope of the invention. The examples refer to the figures in which:

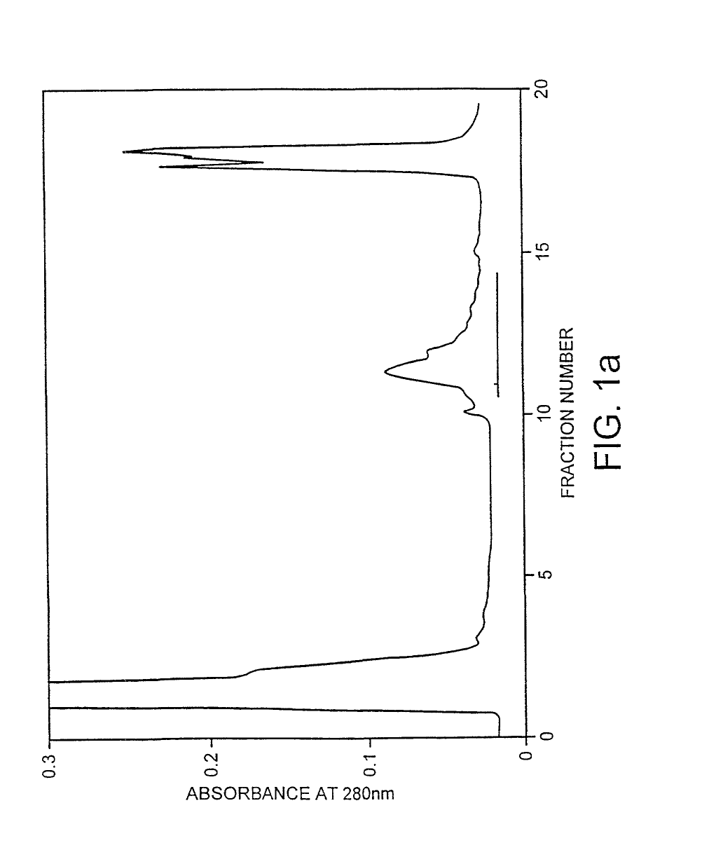

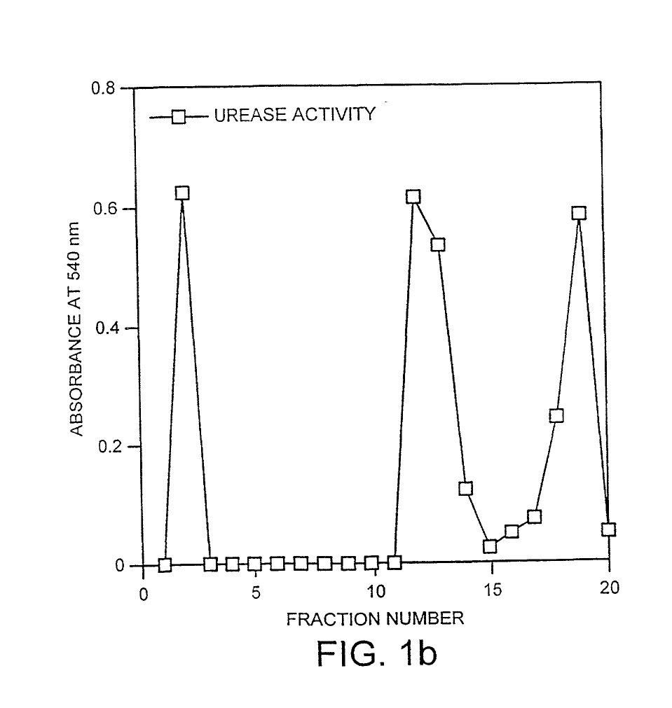

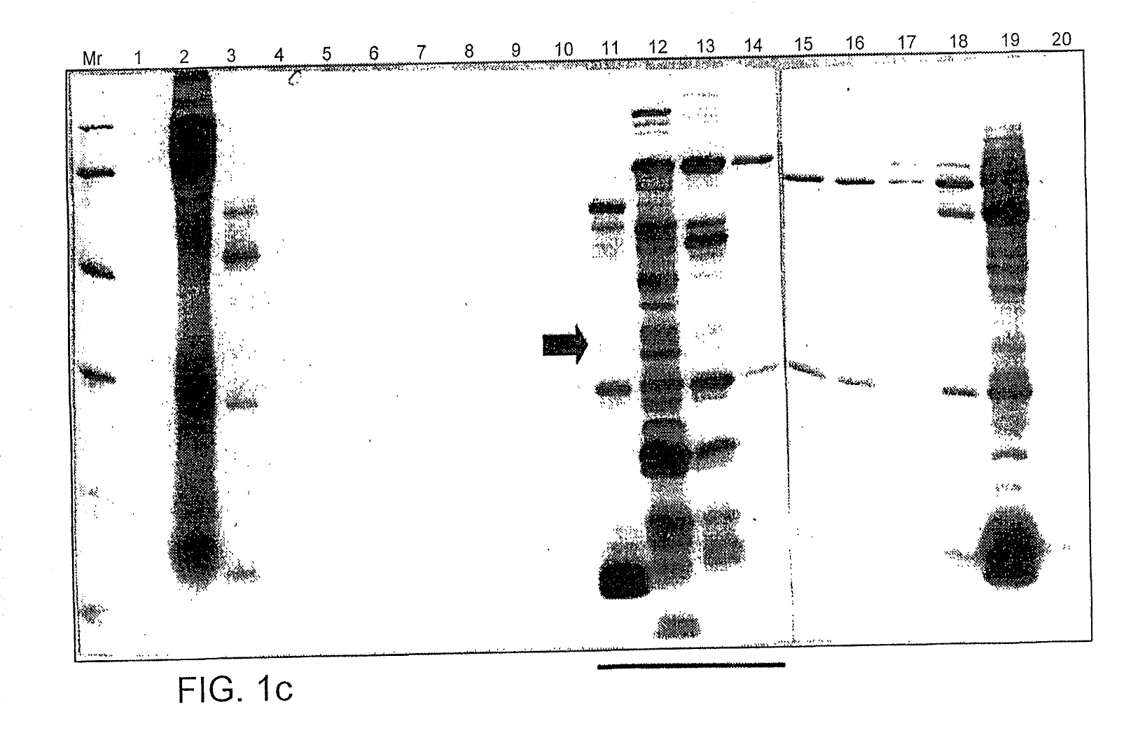

FIG. 1a: shows a typical continuous flow UV absorption profile obtained from Mono Q anion exchange chromatography of concentrated H. pylori sonicate. The bar on the profile represents the fractions collected for further processing (Fractions 11-14);

FIG. 1b: shows a typical urease activity profile of fractions collected from the Mono Q fractionation of H. pylori sonicate. Enzyme activity was determined according to standard methods. Data has been corrected by subtraction of control absorbance values;

FIG. 1c: shows SDS-PAGE analysis of fractions 11-14 collected from the Mono Q column. Arrows indicate the position of the proteins of interest, (fractions 11-13; underscored) containing the 35 kDa antigen. The protein standards are from top to bottom, 94 kDa, 67 kDa, 43, kDa, 30 Kda, 20.1 kDa;

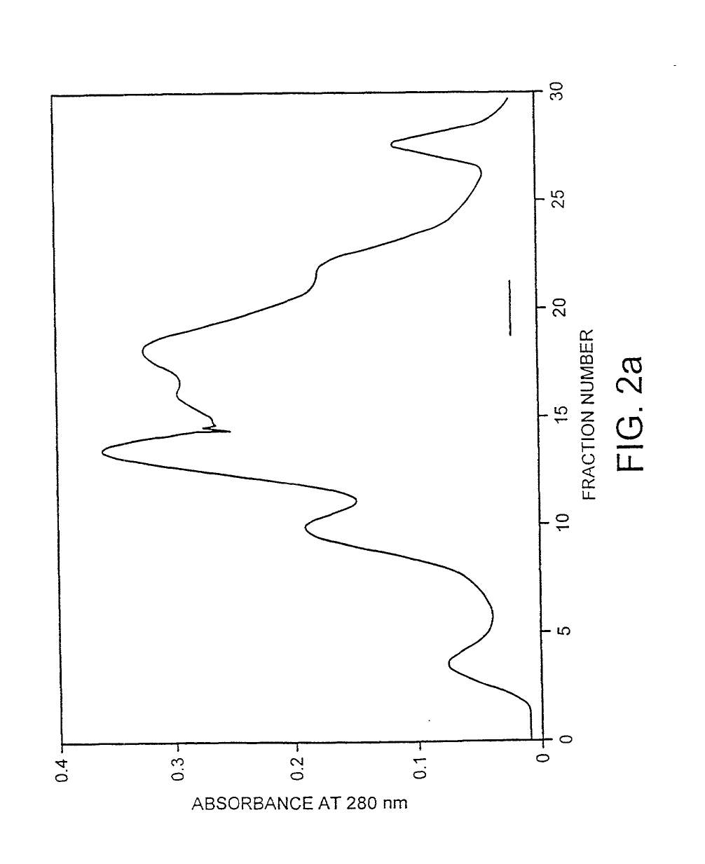

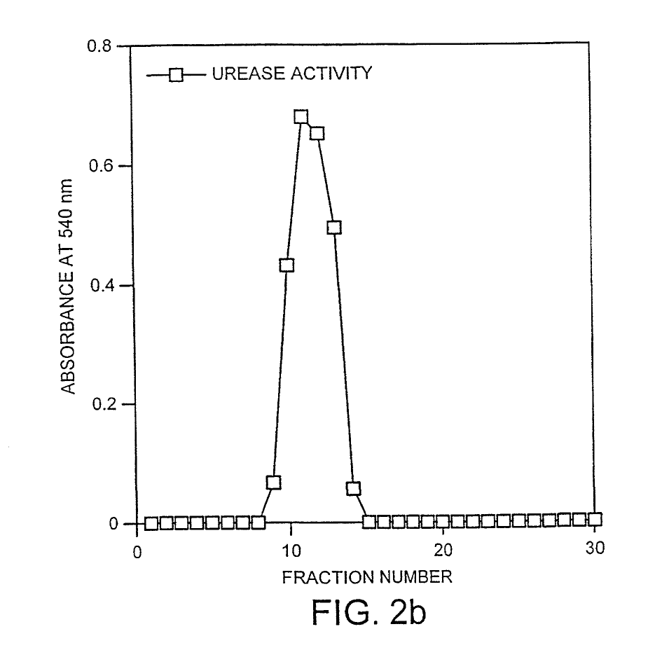

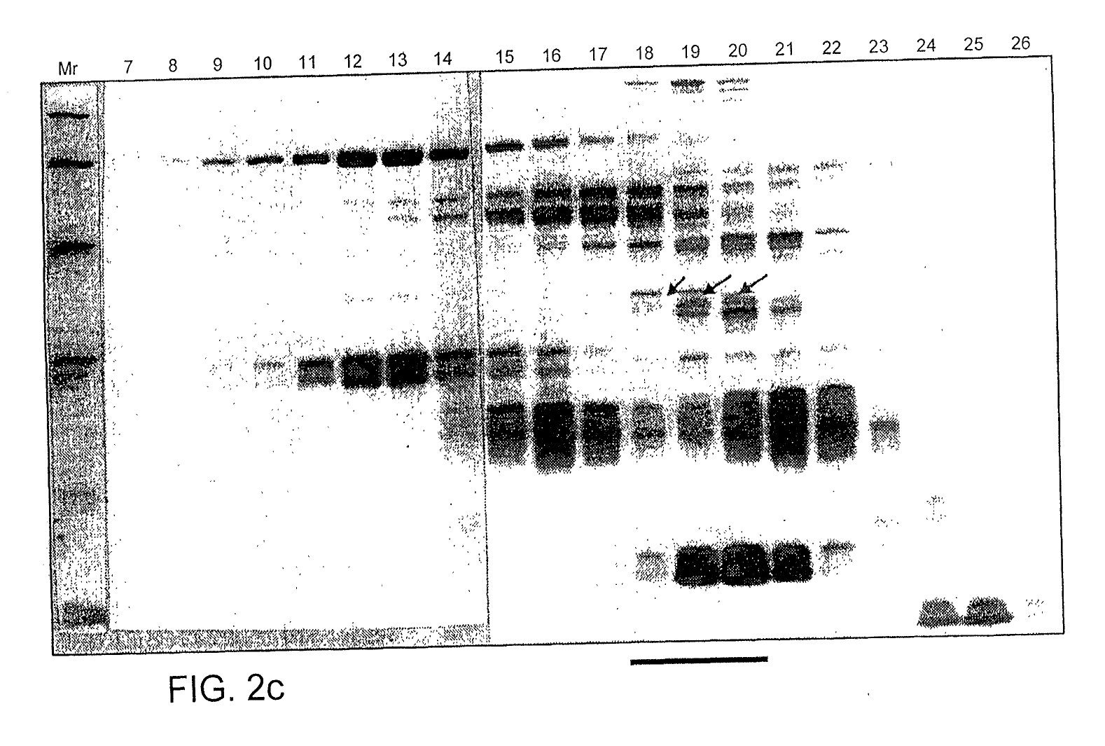

FIG. 2a: shows a typical continuous flow UV absorption profile obtained from Superose 6 FPLC size exclusion chromatography of selected Mono Q fractions as identified in FIG. 1. The bar represents the fractions collected for further processing (Fractions 19-21);

FIG. 2b: shows a typical profile of urease activity in fractions collected following superose 6 FPLC fractionation of the proteins collected in fractions 11-13 from the Mono Q column;

FIG. 2c: shows an SDS-PAGE analysis of fractions collected following Superose 6 FPLC. The 35 kDa protein is present in fractions 18-21 as indicated by the underscore and arrows. Molecular weight standards are from top to bottom, 94 kDa, 67 kDa, 43, kDa, 30 Kda, 20.1 kDa;

FIG. 3: shows an SDS-PAGE analysis of the final purified 35 kDa protein from H. pylori. The molecular standards are as marked;

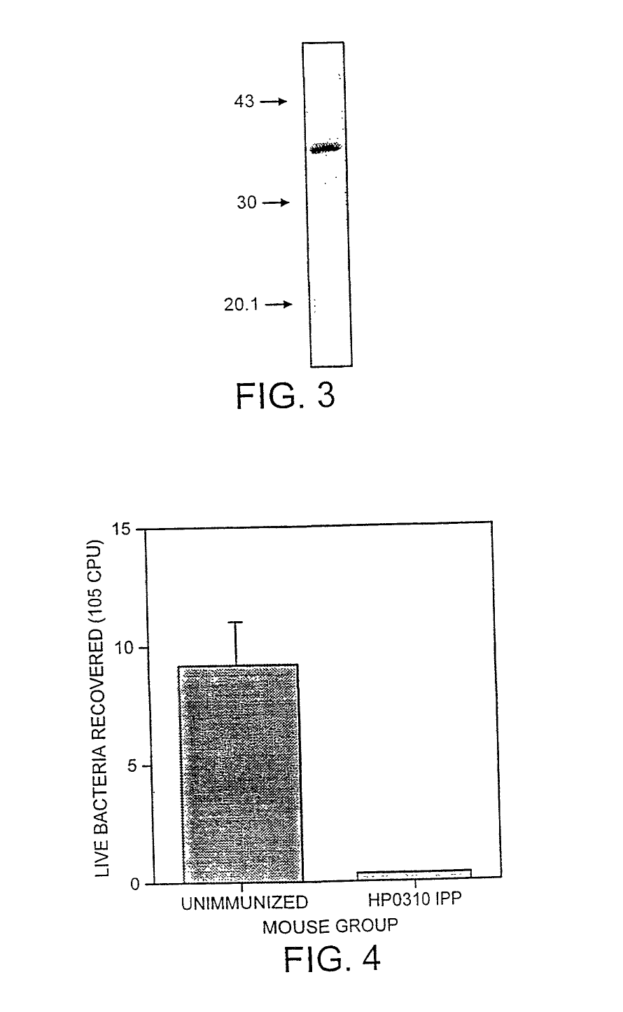

FIG. 4: shows live bacteria recovered (mean) for each group of mice, either unimmunizes or immunised with HP0310 IPP;

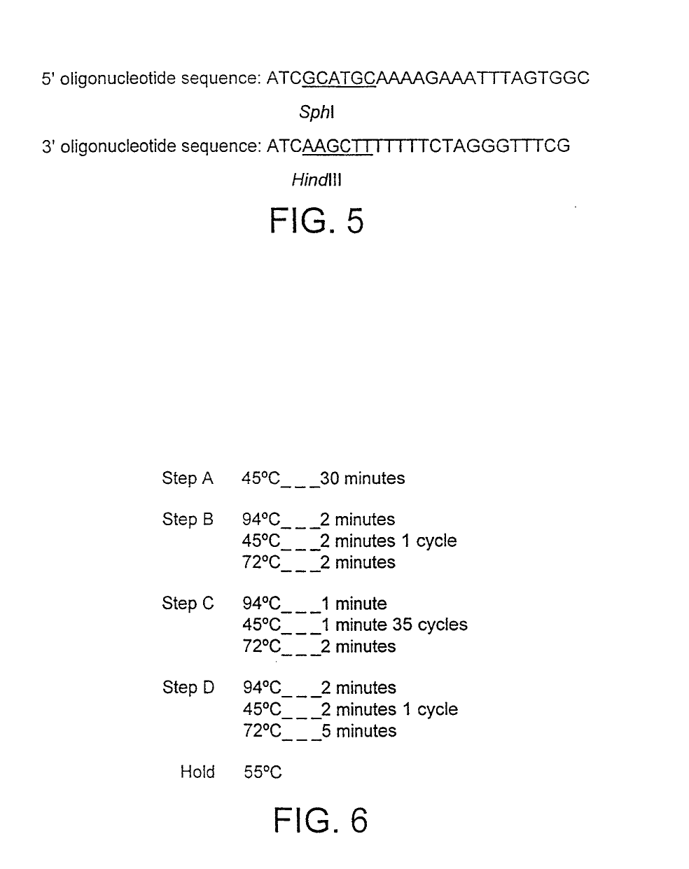

FIG. 5: shows Oligonucleotide sequences for PCR amplification and cloning of the HP0310 gene;

FIG. 6: shows the RT-PCR amplification protocol;

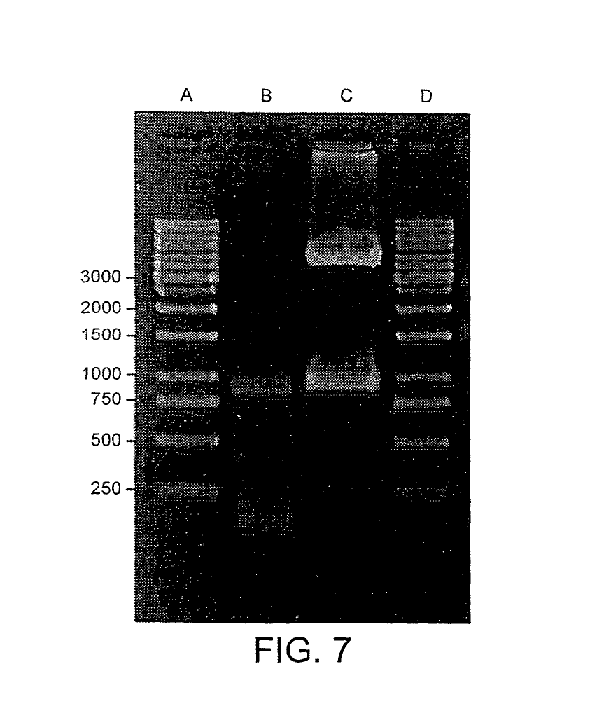

FIG. 7: shows an agarose gel of the HP0310 gene PCR product (B) and the cloned fragment (C) in the cloning vector pCR 2.1; and

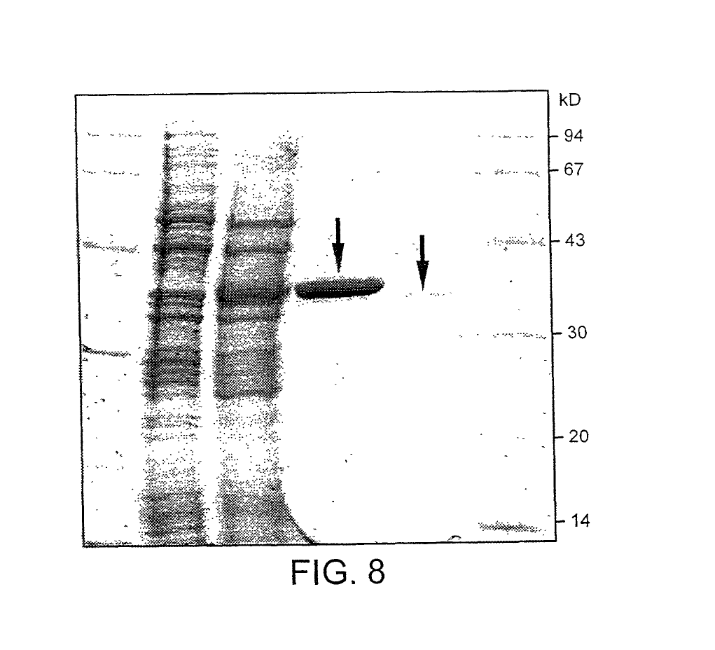

FIG. 8: shows a 12% SDS-PAGE of the expression of the recombinant HP0310 protein. (A) Control E. coli protein profile, (B) Recombinant E.coli expressing the HP0310 antigen, (C) Purified recombinant HP0310, and (D) purified native HP0310. Note the size difference in the recombinant HP0310 is due to the presence of the his-tag. The molecular weight markers are as indicated.

### EXAMPLE 1

1. Identification and Isolation of HP0310 antigen from H.pylori

1.1 Methods

Bacterial Cell Culture.

Helicobacter pylori strain NCTC 11637 was cultured on Chocolate agar plates, then harvested, washed and resuspended in PBS buffer (pH 7.2).

Protein Purification. The H.pylori cell suspension was subjected to sonication using a Sanyo Soniprep 150 ultrasonic disintegrator with a 9.5 mm probe. The sonic amplitude level was set at 6_microns and the machine was operated using 25 cycles of 30 sec on and 60 sec off regulated by an MSE process timer. The sonicated preparation was centrifuged at 10,000 g for 10 min and the supernatant filtered through 0.45 and 0.22 _m filters._The sonicate supernatant was partially purified by anion-exchange FPLC on a Mono Q HR 10/10 column (Pharmacia Biotech Ltd, Uppsala, Sweden) using 0.05 M Tris buffer pH 8.2 and a two-step gradient of Tris buffer containing 0.24 M NaCl and 1.0 M NaCl. Fractions containing the 50/52 kDa protein were pooled, concentrated, and subjected to gel filtration FPLC on a Superose 6 column (Pharmacia Biotech Ltd, Uppsala, Sweden). Elution was with a 0.05 M Tris buffer (pH 7.2). Fractions containing the 50/52 kDa protein were pooled, and further purification was obtained by low pressure liquid chromatography on DEAE-Sepharose CL6B (Pharmacia Biotech Ltd, Uppsala, Sweden) and ceramice hydroxyapatite (Biorad Laboratories, Sydney, Australia).

Briefly, the pooled Superose 6 fractions were loaded onto a small (2.5 ml) column of DEAE-Sephadex CL6B equilibrated with 50 mM Tris buffer (pH 7.4), thoroughly washed with this buffer, then eluted with sequential step gradients comprising 50 mM Tris (pH 7.4) supplemented with 25, 50 and 75 mM NaCl. The final step gradient-eluted material was subsequently loaded onto a small (2.5 ml) column of ceramic hydroxyapatite equilibrated with 5 mM sodium phosphate buffer (pH 7.4). The 35 kDa subunit protein is collected in the initial wash-thorough from this column. Protein fractionation on all chromatography columns employed was monitored continuously at 280 nm and collected fractions were assayed for urease activity. and subjected to analysis by polyacrylamide gel electrophoresis (PAGE). Fractions containing the purified 35 kDa subunit protein were pooled, exhaustively dialyzed against PBS buffer (pH 7.2) and stored at −70° C. until required.

Protein Estimation. Total protein concentrations were determined using the BCA protein assay kit (Pierce, Rockford, Ill., U.S.A).

Polyacrylamide Gel Electrophoresis (PAGE). Fractions or purified 35 kDa subunit protein were assessed for purity by discontinuous SDS-PAGE (5% stack, 12% slab), under either reducing or non-reducing conditions, or by native PAGE (8-25% gradient) analysis.

Amino Acid Sequencing. Purified 35 kDa subunit protein was transferred to polyvinylidene difluoride (PVDF) membrane (BioRad, Sydney, Australia); all buffers used in this process were supplemented with 0.1 mM thioglycolic acid (Sigma, St Louis, Mo., U.S.A.). The transfer membrane was stained with arnido black (Sigma, St Louis, Mo., U.S.A.), then destained and subunit protein bands subsequently excised. N-terminal amino acid sequencing was performed at the Newcastle Protein sequencing facility (Newcastle Protein, The University of Newcastle).

1.2 Results

1.3

This study describes the successful purification of a subunit protein having molecular weight of 35 kDa from the pathogen H.pylori. This protein has been purified from a modification of the protocol used for the preparation of a crude reactive antigen fraction that has been successfully developed as a point-of-care immunodiagnostic kit for detection of H.pylori infection in patients. Typical protein elution, urease activity and reducing SDS-PAGE profiles of fractions collected from both MonoQ and Superose 6 FPLC columns are presented in FIGS. 1 (a-c) and 2(a-c), respectively.

Fractions selected and pooled following gel filtration on Superose 6 are known to contain urease as a component, which has previously been shown to elicit an immunoprotective response and effect eradication of the pathogen in a murine experimental model. Subsequent fractionation by anion exchange chromatography on DEAE-Sepharose CL6B effectively eliminates urease in the protein pool that is eluted at 75 mM NaCl, as determined by SDS-PAGE analysis and urease activity assay (data not shown). Elution of this protein pool once applied to ceramic hydroxyapatite separates the 35 kDa subunit protein from other contaminating proteins present in a single step. Urease activity was not detected in these fractions using the standard assay, nor following prolonged incubation to 24 hours (data not shown). Identical results were obtained with 35 kDa subunit protein following exhaustive dialysis against PBS buffer (pH 7.2) and concentration with crystalline polyethyleneglycol (PEG). Silver staining of the 35 kDa subunit protein preparation on SDS-PAGE following further concentration by centrifugation through Centricon-30 (Amicon, Beverly, Mass., U.S.A.) did not reveal the presence of either of the urease subunit components.

The purified 35 kDa protein has been further assessed on denaturing PAGE under both reducing and non-reducing conditions. Analysis by denaturing PAGE indicates that this protein exists as a discrete 35 kDa subunit protein under both reducing and non-reducing conditions (FIG. 3).

The purified 35 kDa subunit protein was identified following N-terrninal sequencing at the Newcastle Protein facility. The sequence data obtained for the first 12 amino acid residues corresponding to the purified 35 kDa subunit band observed on reducing SDS-PAGE was AKEILVAYGVDI. Preliminary identification of this protein was obtained by BLAST (Basic Local Alignment Sequence Tool) analysis of this sequence using the Swiss-Prot on-line database and the genomic database for the H.pylori strain 26695 at T.I.G.R. These combined analyses revealed that this protein corresponds to a predicted conserved hypothetical protein designated HP0310. As yet the function of this protein is unknown about this protein since (i) it has never been purified in other laboratories and (ii) comparative sequence analysis reveals homology with proteins having diverse functions.

The data below aligns the sequences for the native NCTC 11637 strain protein with the predicted sequence for this protein in strain 26695 and the sequence determined for the recombinant NCTC 11637 protein cloned by Dr Richard McCoy in this laboratory. The BLAST analysis was obtained using the predicted sequence of HP0310 from strain 26695: significant matches only are shown (i.e. P(N)<0.001). Alignments for the top 3 matches yield no insight concerning the functional identity or significance for the purified 35 kDa protein which has regions of sequence homology corresponding to (i) a hypothetical protein from Synechocystis sp., (ii) the nodulation protein (nodB) from Bacillus stearothermophilus and (iii) a hypothetical protein in Bacillus strearothermophilus.

| SEQUENCE ALIGNMENT | |
|---|---|
| Native AKEILVAYGVDI | |
| Recomb: AKEILVAYGVDIDAVAGWLGSYGGEDSPDDISRGLFAGEVGIPRLLKLFKKYHLPATWF | |
| 26695: MAKEILVAYGVDIDAVAGWLGSYGGEDSPDDISRGLFAGEVGIPRLLKLFKKYHLPATWF | 60 |
| Recomb: PGHSIETFPEQMKMIVDAGHESGKSIELIKDLTGKAP | |
| 26695: SPGHSIETFSEQMKMIVDAGHEVGAHGYSHENPIAMTAKQEEDVLLKSVELIKDLTGKAP | 120 |
| Recomb: QAMWRRGGKFSNITNELRLKHGFKYSLEAKDWMKP | |
| 26695:TGYVAPWWEFSNITNELLLKHGFKYDHSLMHNDFTPYYVRVGDSWSKIDYSLEAKDWNKP | 180 |
| Recomb: IRGVDVAPMMFIKKSPNSFGFVSPHDIGQMWIDQFDWVYREMDYA | |
| 26695: LIRGVETDLVEIPANWYLDDLPPMMFIKKSPNSFGFVSPHDIGQMWIDQFDWVYREMDYA | 240 |
| Recomb: VFSMTIHPDVSARPQVLLMHEKIIEHINKHEGVRWVTFNEIADDFLKRNPRKK | |
| 26695: VFSMTIHPDVSARPQVLLMHEKIIEHINKHEGVRWVTFNEIADDFLKRNPRKK | 293 |

| | | |
|---|---|---|
| BLAST ANALYSIS | | |
| Query=MySequence (293 letters) | | |
| High Probability Sequences producing High-scoring Segment | | |
| Pairs: | | |
| Score | P(N) | N |
| gi\|2313406\| (AE000549) conserved hypothetical . 1590 | 5.0e-212 | 1 |
| gnl\|PID\|d1018374 (D90907) hypothetical protein [Sy.. 184 | 1.4e-16 | 1 |
| pir\|\|B47692 nodulation protein nodB homolog[13 Ba. . . 132 | 2.4e-09 | 1 |
| sp\|Q04729\| YFU2_BACST HYPOTHETICAL 30.6 KD 132 | 2.4e-09 | 1 |
| gi\|2626811\| (D83967) YfjS [Bacillus subtilis]>g.. .125 | 2.3e-08 | 1 |
| gnl\|PID\|e325402 (Z97209) hypothetical protein [Schiz . . . 96 | 1.3e-07 | 2 |
| gnl\|PID\|e1185261 (Z99112) alternate gene name: ymxI; .112 | 1.5e-06 | 1 |
| sp\|P50850\|YLXY_BACSU HYPOTHETICAL 31.5 KD .. 105 | 1.4e-05 | 1 |
| gi\|2612282\| (AF015825) NodB-like protein [Bacill . . . 95 | 0.00034 | 1 |
| gnl\|PID\|e325211 (Y14082) hypothetical protein [Bacil . . . 78 | 0.00061 | 3 |
| gnl\|PID\|e1251975 (AL021897) hypothetical protein .93 | 0.00065 | 1 |

```
SEQUENCE HOMOLOGY
1.
>gnl|PID|d1018374 (D90907) hypothetical protein [Synechocystis sp.]
Length = 335
  Score = 184 (85.0 bits), Expect = 1.4e − 16, P = 1.4e − 16
  Identities = 39/104 (37%), Positives = 58/104 (55%)
```

| | |
|---|---|
| Query: 42 GIPRLLKLFKKYHLPATWFSPGHSIETFSEQMKMIVDAGHEVGAHGYSHENPIAMTAXQE | 101 |
| G+PR+L L KY + T G ++E ++10 ++ K IV GHE AHG+ +N MTA QE | |
| Sbjct:95 GVPRILDLLDKYKIKITSHMSGRTVEMYPDRAKEIVQRGHEAAAHGWDWDNEFNMTAPQE | 154 |
| Query: 102 EDVLLKSVELIKDLTGKAPTGYVAPWWEFSNITNELLLKHGFKY | 145 |
| D + ++V++I +TG+ GY AP S +L + GF Y | |
| Sbjct: 155 RDFIQRNVDIILKVTGQRAVGYNAPGLRGSVNILTVLNELGFVY | 198 |
| 2. | |

```
>pir//B47692 nodulation protein nodB homolog—Bacillus stearothermophilus
                        Length = 265
  Score = 132 (61.0 bits), Expect = 2.4e − 09, P = 2.4e − 09
  Identities = 28/82 (34%), Positives = 49/82 (59%)
```

| | |
|---|---|
| Query: 45 RLLKLFKKYHLPATWFSPGHSIETFSEQMKMIVDAGHEVGAHGYSHENPIAMTAKQEEDV | 104 |
| ++L + KK+ + AT+F GH ++T + +K +V GH VG H +SH + ++A + + | |
| Sbjct: 84 KILDVLKKHDVHATFFVTGHYLKTAPDLVKRMVKEGHIVGNHSWSHPDMTTISADKIKKE | 143 |
| Query: 105 LLKSVELIKDLTGKAPTGYVAP | 126 |
| L + +K+LTG+ T YV P | |
| Sbjct: 144 LDAVSDKVKELTGQEGTVYVRP | 165 |
| 3. | |

```
>sp|Q04729|YFU2_BACST HYPOTHETICAL 30.6 KD PROTEIN IN FUMA 3′REGION PRECURSOR
                        (ORF2) >gi|551706| (L05611) [fumA(Bst)] gene products [Bacillus
                        stearothermophilus]
                        Length = 265
  Score = 132 (61.0 bits), Expect = 2.4e − 09, P = 2.4e − 09
  Identities = 28/82 (34%), Positives = 49/82 (59%)
```

| | |
|---|---|
| Query: 45 RLLKLFKKYHLPATWFSPGHSIETFSEQMKMIVDAGHEVGAHGYSHENPIAMTAKQEEDV | 104 |
| ++L + KK+ + AT+F GH ++T +K +V GH VG H SH ++A + + | |
| Sbjct: 84 KILDVLKKHDVHATFFVTGHYLKTAPDLVKRMVKEGHIVGNHSWSHPDMTTISADKIKKE | 143 |
| Query: 105 LLKSVELIKDLTGKAPTGYVAP | 126 |
| L + +K+LTG+ T YV P | |
| Sbjct: 144 LDAVSDKVKELTGQEGTVYVRP | 165 |

2. Testing the Native H.pylori Protein HP0310 isolated from NCTC strain 11637, as a vaccine antigen

2.1 Methods

Immunization of mice

The antigen was tested in a mouse H.pylori infection model using prophylactic immunization.

Female, specific pathogen free C57BL/6 mice were obtained from the Central Animal House at the University of Newcastle, NSW, Australia. Animal experiments were performed with the approval of the Animal Care and Ethics Committee of The University of Newcastle and mice were housed five per cage in isolator cages. Mice were immunized by the intra-Peyer's patch (IPP) route to test the efficacy of the antigen as a vaccine candidate as this immunization route has been shown to give a maximal intestinal immunization (1,2) and is therefore useful for screening proteins which have potential as oral vaccine antigens. The antigen HP0310 (at 0.5 mg protein/mL) was contained in an homogenate of equal quantities of PBS and Freund's incomplete adjuvant. For IPP immunization each mouse was anaesthetised by intraperitoneal injection of 200 μL of a ketamine (Parnell Laboratories, Australia), xylazine (Bayer) mixture made by mixing 10 mL of ketamine (100 μ/ml) and 1 ml of xylazine (100 μg/mL), the abdomen shaved and swabbed with 70% alcohol and a midline incision made in the skin and muscle layers to expose the intestine. Visible Peyer's patches were located along the length of the intestine and approximately 3 μL of homogenate injected directly under the serosa of each Peyer's patch. The muscle and skin layers were sutured and the mouse kept warm until recovery from anaesthesia. For each experiment, ten mice were immunized and another 10 mice left untreated as the unimmunized controls.

Infection of mice with H.pylori

Mice were infected two weeks after immunization. H.pylori Sydney strain 1 (SS1) was obtained from Prof. A. Lee, The University of NSW, Sydney Australia. This strain of H.pylori has been shown to successfully colonise the stomachs of C57BL/6 mice (3). The H.pylori was grown on chocolate agar plates for 3 days in a microaerophilic 37° C. incubator and harvested into PBS. The concentration of H.pylori was determined from the optical density reading at 405 nm and a regression curve relating optical density to H.pylori concentration. Mice were infected, by gavage, on three successive days with a 100 μL volume containing approximately 10⁸ H.pylori., and actual concentration of live H.pylori was determined by culture of serial ten-fold dilutions of the live H.pylori preparation on chocolate agar for three days. The actual dose of live H.pylori was therefore calculated retrospectively. The doses on the three successive days were: 2.0×10⁸, 5.0×10⁸, 1.0×10⁸.

Sample collection

Four weeks after infection the mice were killed by intraperitoneal pentobarbitone overdose and the stomachs removed. The stomachs were cut in half longitudinally and one half was homogenised in 1 mL of PBS and aliquots of serial dilutions plated out on chocolate agar plates and cultured for 3 days. Colonies were counted to determine the number of colony forming units (CFU) of H.pylori in the half stomach of each mouse. The mean ± SEM was calculated for each group.

2.2 Results

Table 1 and FIG. 4 show the mean recovery of live bacteria from the half stomachs of each group of mice.

TABLE 1
Bacteria recovered from homogenised half stomach

| | | Mean CFU | | |
|---|---|---|---|---|
| Group | Number of mice | (10⁵) | SD | SEM |
| Non-immunized | 10 | 9.3 | 5.8 | 1.8 |
| HP0310 IPP | 9 | 0.30 | 0.19 | 0.06 |

Unpaired “t test” comparison of the groups shows that the mean CFU is significantly lower in the group immunized with HP0310 (P<0.001). The percentage clearance of bacteria when the immunized group is compared to the unimmunized group is 97%.

Conclusion

The protein HP0310 from H.pylori strain NCTC 11637 is a protective antigen when used prophylactically to prevent H.pylori infection in mice. It is anticipated that this protein would also be effective in a therapeutic vaccine.

3. Cloning and Expression of the H.pylori NCTC 11637 HP0310 Gene

3.1 Introduction

The HP0310 protein from the H.pylori NCTC 11637 strain was first noted in protein analysis on the soluble fraction of sonicated bacterial preparations. The protein was identified by comparing amino acid sequence obtained from the isolated protein with the TIGR H.pylori genome database. Immunization and challenge studies using the purified native protein indicated induction of appreciable protection and warranted the attempt to clone the gene for the production of recombinant protein

3.2 Methods & Results

Oligonucleotides: Oligonucleotides were designed for the 5′ and 3′ ends of HP0310 directly from the TIGR database HP0310 sequence of H.pylori strain 26695 (FIG. 5). To accommodate later cloning of the amplified gene into an expression plasmid vector, a restriction enzyme site was engineered into the 5′ end of each oligonucleotide. The selected enzyme sites, SphI and HindIII for the 5′ and 3′ primers respectively, were selected after performing a enzyme site search on the HP0310 sequence of H.pylori strain 26695 using an appropriate software package and in consideration for the available enzyme sites in the multiple cloning site of pQE30 series vectors

RNA production: Total RNA was made from a 3 day culture of H. pylori NCTC 11637 strain by using the Boehringer Mannheim High Pure RNA Isolation Kit. The standard procedure for isolation of RNA from bacteria as outlined in the kit protocol was followed and included treatment with DNase I. The isolated RNA was made to a final volume of 50 μl in DEPC treated distilled deionized water (dd.H₂O).

cDNA production: To produce cDNA from the isolated RNA, 5 μl of total RNA was mix with 2 μl of each oligonucleotide primer (at approximately 0·5 μg/μl), 2 μl of dNTP mix containing 2·5 mM of each dNTP, 5 μl of 5X reaction buffer (Promega), 3 μl of 1 mg/ml bovine serum albumin, 10 units of RNasin (Promega), and 200 units of Moloney murine leukaemia virus reverse transcriptase (Promega). The volume was made up to 25 μl with dd.H₂O and incubated at 42° C. for 60 minutes. The reaction was stopped by incubation at 70° C. for 10 minutes and the final volume made up to 50 μl with dd.H₂O.

Polymerase chain reaction amplification: Amplification was performed on 5 μl of the cDNA product using Taq DNA polymerase (Promega) and MgCl₂ concentrations of 1, 3 and 5 mM. PCR reaction mixes were made up to 50 μL with dd.H₂O and pulsed in a microfuge before amplification. PCR reactions were performed in a Hybaid Touchdown thermal cycler using the protocol as outlined in FIG. 6. Upon completion of the amplification, reaction tubes were transferred to 4° C. and 10 μL of each reaction run on a 1% agarose (Progen, Australia) gel electrophoresis. The agarose gel was stained with ethidium bromide and inspected for a band at approximately 900 base pairs when compared to a 1 kilobase pair ladder (Progen) (FIG. 7).

PCR fragment purification and cloning: Upon identification of a successful amplification reaction, i.e. a reaction containing a fragment of the predicted size, the PCR product was purified using a purification kit (Boehringer Mannheim). The purified product was then excised from a 1% agarose gel and the fragment purified using a Progen Band Pure purification kit. The isolated fragment was then ligated into the pCR2·1 plasmid vector as supplied with the Original TA Cloning kit (Invitrogen, U.S.A.). Ligation mix was transformed into competent TOP10F′ E. coli strain and plated onto LB agar plates containing 100 μg ampicillin/milliliter and overlayed with agar containing 1 mM IPTG (Progen) and 0·02% X-gal (Amresco, U.S.A.). The plates were examined for colonies showing a lack of β-galactosidase activity indicating insertion of the fragment into the pCR2·1 vector and half a dozen of these were selected for plasmid DNA preparation using the Pharmacia Flexiprep system. The isolated clones' plasmid DNA were digested with EcoRI to excise the inserted fragment and examined on a 1% agarose gel (FIG. 7). Clones containing the correct size fragment were then sent for nucleotide sequencing using an ABI Prism 377 DNA automated sequencer at the DNA sequencing section, Newcastle Biomedical Research Facility, Newcastle University, Australia.

Cloning into pQE expression vector: The cloned NCTC 11637 HP0310 gene was excised from the pCR2·1 vector using the SphI and HindIll restriction enzyme sites engineered into the PCR primers. The fragment was ligated into the corresponding sites in the pQE31 expression vector multiple cloning site and transformed into competent JM109 E. coli strain. Colonies were grown on LB ampicillin plates and again half a dozen possible clones selected for plasmid DNA analysis. Cloning was confirmed by restriction enzyme analysis and sequencing. Upon confirmation of the cloning, two clones were selected and cultures grown in LB broth for glycerol storage at −70° C.

Expression of recombinant HP0310 protein: Expression from the pQE series vectors is under the control of the T5 promoter with two lac operator sequences. To express the cloned HP0310 gene, the pQE31-HP0310 plasmid clone was transformed into M15 E. coli strain cells which contain the pREP4 plasmid. The pREP4 plasmid provides the lac repressor gene which is used control expression of the inserted gene. Transformation was confirmed by plasmid DNA analysis and then a fresh plate of colonies made on LB agar containing 100 μg ampicillin/mL and 25 μg kanamycin/mL (LBA/AK), the kanamycin resistance gene being carried by the pREP4 plasmid. A single colony of the expression clone in M15 cells was inoculated into 5 mLs LB broth containing ampicillin and kanamycin (LB/AK) and grown overnight at 37° C. 0·5 mLs of the overnight culture was used to seed 4·5 mLs of fresh LB/AK broth and this culture grown at 37° C. for 2 hours. Gene expression was induced by adding 100 mM sterile IPTG to a final concentration of 2 mM and the culture re-incubated at 37° C. for a further 4 hours.

After expression incubation was completed, cells were centrifuged at 3000 rpm in a Beckman GPR bench top centrifuge for 10 minutes at 10° C. and the supernatant discarded. Cells were resuspended in 2·5 mLs of 8 M urea in 0·1 M sodium dihydrogen phosphate and 0.01 M Tris, pH 8·0, lysis buffer. The cell suspension was sonicated on ice at an amplitude of 7 microns for four cycles of 20 seconds with sonication followed by 20 seconds with no sonication using a Sanyo Soniprep 150 sonicator with a 3 mm diameter probe under the control of an MSE process timer. Sonicate preparations were centrifuged as before for 15 minutes and the supernatant transferred to a fresh tube. Pellets were resuspended in 1 ml of PBS. 10 μL of each of the supematant and pellet preparations were added to an equal volume of PAGE reducing loading buffer containing 4% SDS and electrophoresed on a 12% acrylamide mini Ready Gel with a 4% acrylamide stacking layer (Bio Rad, U.S.A.). The gel was run at 80 volts for approximately 15 minutes and then at 180 volts until the bromophenol blue marker dye. The resulting gel was stained in 0·1% Coomassie blue stain and examined for recombinant protein which should have been at approximately 35 kDa (FIG. 8).

References

1. Dunkley, M. L. and Husband, A. J. (1986) The induction and migration of antigen-specific helper cells for IgA responses in the intestine. Immunology 57, 379-385.

2. Cripps, A. W., Dunkley, M. L., and Clancy, R. L. (1994) Mucosal and systemic immunizations with killed Pseudomonas aeruginosa protect against acute respiratory infection in rats. Infection and Immunity 62, 1427-1436.

3. Lee, A., O'Rourke, J., Ungria, M. C. D., Robertson, B., Daskalopoulos, G., and Dixon, M. F. (1997) A standardized mouse model of Helicobacter pylori infection: Introducing the Sydney Strain. Gastroenterology 112, 1386-1397.

1. An H.pylori antigenic protein, having a molecular weight of 35 kDa, as measured by SDS-PAGE under reducing or non-reducing conditions and having the amino acid sequence:
MAKEILVAYGVDIDAVAGWLGSYGGEDSPDDISRGLFAGEVGIPRLLKLF
KKYHLPATWFSPGHSIETFSEQMKMIVDAGHEVGAHGYSHENPIAMTAKQ
EEDVLLKSVELIKDLTGKAPTGYVAPWWEFSNITNELLLKHGFKYDHSLM
HNDFTPYYVRVGDSWSKIDYSLEAKDWMKPLIRGVETDLVEIPANWYLDD
LPPMMFIKKSPNSFGFVSPHDIGQMWIDQFDWVYREMDYAVFSMTTHPDV
SARPQVLLMHEKIIEHINKHEGVRWVTFNEIADDFLKRNPRKK.

2. A protein as claimed in claim 1 provided in substantially pure form, preferably substantially free of other proteins.

3. An antigenic/immunogenic homologue or derivative of a protein as claimed in claim 1 or claim 2.

4. One or more antigenic/immunogenic fragments of a protein as defined in claim 1 or claim 2, or of a homologue or derivative as defined in claim 3.

5. A recombinant nucleic acid molecule comprising or consisting of:
(i) the sequence:
ATGGCAAAAGAAATTTTAGTGGCTTATGGTGTGGATATTGATGCGGTGGC
TGGTTGGTTAGGGAGCTATGGTGGGGAGGATTCGCCTGATGATATTTCGC
GCGGGCTTTTTGCGGGTGAAGTGGGGATCCCACGGCTTTTGAAATTGTTT
AAAAAATACCATCTCCCGGCGACTTGGTTTTCGCCGGGGCATTCTATTGA
AACTTTCTCTGAACAAATGAAAATGATCGTGGATGCAGGGCATGAAGTGG
GCGCGCATGGGTATTCGCATGAAAACCCTATCGCTATGACGGCCAAGCAA
GAAGAAGACGTTTTGTTAAAAAGCGTTGAGTTGATTAAAGATCTCACCGG
CAAGCCCCCACAGGCTATGTGGCGCCGTGGTGGGAGTTTTCTAATATCAC
TAATGAATTGCTTTTAAAACACGGCTTCAAATACGACCACTCGCTCATGC
ACAATGATTTCACGCCCTATTATGTGCGCGTGGGGGATAGTTGGAGCAAG
ATTGATTATAGTTTGGAAGCTAAGGATTGGATGAAGCCTTTAATCCGTGG
GGTGGAAACCGATCTGGTGGAAATCCCTGCGAACTGGTATTTGGACGATT
TACCGCCGATGATGTTCATCAAAAAGTCCCCCAATAGTTTTGGTTTTGTA
AGTCCGCACGATATAGGGCAAATGTGGATCGATCAATTTGATTGGGTTTA
TCGTGAGATGGATTATGCGGTGTTTAGCATGACAATCCACCCTGATGTGA
GCGCCCGTCCGCAAGTGTTGCTCATGCATGAAAAAATCATTGAGCATATC
AACAAGCACGAGGGCGTGCGTTGGGTAACATTCAATGAAATCGCTGATGA
TTTCTTAAAACGAAACCCTAGAAAAAAA.;
(ii) a sequence which is complementary to the sequence in (i);
(iii) a sequence which codes for the same protein, as those sequences of (i) or (ii);
(iv) a sequence which has substantial identity with any of those of (i), (ii) and (iii);
(v) a sequence which codes for a homologue, derivative or fragment of the protein as described herein.

6. A vector comprising a nucleic acid sequence as defined in claim 5.

7. A host cell containing a vector as defined in claim 6.

8. An immunogenic/antigenic composition comprising a protein as defined in claim 1 or claim 2, and/or a homologue or derivative as defined in claim 3, and/or one or more fragments as defined in claim 4.

9. An immunogenic/antigenic composition as claimed in claim 8 which is a vaccine or is for use in a diagnostic assay.

10. A vaccine composition comprising one or more nucleic acid sequences as defined in claim 5.

11. A method for the detection/diagnosis of H.pylori which comprises the step of bringing into contact a sample to be tested with a protein as defined in claim 1 or claim 2, a homologue or derivative as defined in claim 3, or a fragment thereof, as defined in claim 4.

12. A method as claimed in claim 11 wherein the sample is a biological sample, such as a tissue sample or a sample of blood or saliva obtained from a subject to be tested.

13. An antibody capable of binding to a protein as defined in claim 1 or claim 2, a homologue or derivative as defined in claim 3 or a fragment as defined in claim 4.

14. A method for the detection/diagnosis of H.pylori which comprises the step of bringing into contact a sample to be tested with at least one antibody as defined in claim 13

15. A method for the detection/diagnosis of H.pylori which comprises the step of bringing into contact a sample to be tested with at least one nucleic acid sequence as defined in claim 5.

16. A method as claimed in claim 15 wherein the sample is a biological sample, such as a tissue sample or a sample of blood or saliva obtained from a subject to be tested.

17. A method of vaccinating a subject against H.pylori which comprises the step of administering to a subject a protein as defined in claim 1 or claim 2, a derivative or homologue as defined in claim 3, one or more fragments thereof as defined in claim 4, or an immunogenic composition as defined in claim 8 or claim 9.

18. A method of vaccinating a subject against H.pylori which comprises the step of administering to a subject a nucleic acid molecule as defined in clam 5.

19. A method for the prophylaxis or treatment of H.pylori infection which comprises the step of administering to a subject a protein as defined in claim 1 or claim 2, a derivative or homologue as defined in claim 3, one or more fragments thereof as defined in claim 4, or an immunogenic composition as defined in claim 8 or claim 9.

20. A method for the prophylaxis or treatment of H.pylori infection which comprises the step of administering to a subject a nucleic acid molecule as defined in claim 5.

20. A kit for use in detecting/diagnosing H.pylori infection comprising a protein as defined in claim 1 or claim 2, a homologue or derivative as defined in claim 3, one or more fragments thereof as defined in claim 4, or an antigenic composition, as defined in claim 8 or claim 9.

21. A kit for use in detecting/diagnosing H.pylori infection comprising one or more nucleic acid molecules as defined in claim 5.

22. A kit for use in detecting/diagnosing H.pylori infection comprising one or more antibodies as defined in claim 13.

23. The use of a protein as defined in claim 1 or claim 2, a homologue or derivative as defined in claim 3, one or more fragments thereof as defined in claim 4, or an antigenic composition as defined in claim 8 or claim 9 in the manufacture of a medicament for the prophylaxis or treatment of H.pylori infection;

24. The use one or more nucleic acid molecules as defined in claim 5, or one or more fragments thereof in the manufacture of a medicament for the prophylaxis or treatment of H.pylori infection.


SEQUENCE LISTING (27 sequences)

SEQ ID NO: 1 - protein, 293 aa, Helicobacter pylori
MAKEILVAYGVDIDAVAGWLGSYGGEDSPDDISRGLFAGEVGIPRLLKLFKKYHLPATWFSPGHSIETFSEQMKMIVDAGHEVGAHGYSHENPIAMTAKQEEDVLLKSVELIKDLTGKAPTGYVAPWWEFSNITNELLLKHGFKYDHSLMHNDFTPYYVRVGDSWSKIDYSLEAKDWMKPLIRGVETDLVEIPANWYLDDLPPMMFIKKSPNSFGFVSPHDIGQMWIDQFDWVYREMDYAVFSMTIHPDVSARPQVLLMHEKIIEHINKHEGVRWVTFNEIADDFLKRNPRKK

SEQ ID NO: 2 - DNA, 879 bp, Helicobacter pylori
atggcaaaagaaattttagtggcttatggtgtggatattgatgcggtggctggttggttagggagctatggtggggaggattcgcctgatgatatttcgcgcgggctttttgcgggtgaagtggggatcccacggcttttgaaattgtttaaaaaataccatctcccggcgacttggttttcgccggggcattctattgaaactttctctgaacaaatgaaaatgatcgtggatgcagggcatgaagtgggcgcgcatgggtattcgcatgaaaaccctatcgctatgacggccaagcaagaagaagacgttttgttaaaaagcgttgagttgattaaagatctcaccggcaaagcccccacaggctatgtggcgccgtggtgggagttttctaatatcactaatgaattgcttttaaaacacggcttcaaatacgaccactcgctcatgcacaatgatttcacgccctattatgtgcgcgtgggggatagttggagcaagattgattatagtttggaagctaaggattggatgaagcctttaatccgtggggtggaaaccgatctggtggaaatccctgcgaactggtatttggacgatttaccgccgatgatgttcatcaaaaagtcccccaatagttttggttttgtaagtccgcacgatatagggcaaatgtggatcgatcaatttgattgggtttatcgtgagatggattatgcggtgtttagcatgacaatccaccctgatgtgagcgcccgtccgcaagtgttgctcatgcatgaaaaaatcattgagcatatcaacaagcacgagggcgtgcgttgggtaacattcaatgaaatcgctgatgatttcttaaaacgaaaccctagaaaaaaa

SEQ ID NO: 3 - protein, 12 aa, Helicobacter pylori
AKEILVAYGVDI

SEQ ID NO: 4 - protein, 59 aa, Helicobacter pylori
AKEILVAYGVDIDAVAGWLGSYGGEDSPDDISRGLFAGEVGIPRLLKLFKKYHLPATWF

SEQ ID NO: 5 - protein, 60 aa, Helicobacter pylori
MAKEILVAYGVDIDAVAGWLGSYGGEDSPDDISRGLFAGEVGIPRLLKLFKKYHLPATWF

SEQ ID NO: 6 - protein, 37 aa, Helicobacter pylori
PGHSIETFPEQMKMIVDAGHESGKSIELIKDLTGKAP

SEQ ID NO: 7 - protein, 60 aa, Helicobacter pylori
SPGHSIETFSEQMKMIVDAGHEVGAHGYSHENPIAMTAKQEEDVLLKSVELIKDLTGKAP

SEQ ID NO: 8 - protein, 35 aa, Helicobacter pylori
QAMWRRGGKFSNITNELRLKHGFKYSLEAKDWMKP

SEQ ID NO: 9 - protein, 60 aa, Helicobacter pylori
TGYVAPWWEFSNITNELLLKHGFKYDHSLMHNDFTPYYVRVGDSWSKIDYSLEAKDWMKP

SEQ ID NO: 10 - protein, 45 aa, Helicobacter pylori
IRGVDVAPMMFIKKSPNSFGFVSPHDIGQMWIDQFDWVYREMDYA

SEQ ID NO: 11 - protein, 60 aa, Helicobacter pylori
LIRGVETDLVEIPANWYLDDLPPMMFIKKSPNSFGFVSPHDIGQMWIDQFDWVYREMDYA

SEQ ID NO: 12 - protein, 53 aa, Helicobacter pylori
VFSMTIHPDVSARPQVLLMHEKIIEHINKHEGVRWVTFNEIADDFLKRNPRKK

SEQ ID NO: 13 - protein, 53 aa, Helicobacter pylori; identical to SEQ ID NO: 12

SEQ ID NO: 14 - protein, 60 aa, Helicobacter pylori
GIPRLLKLFKKYHLPATWFSPGHSIETFSEQMKMIVDAGHEVGAHGYSHENPIAMTAKQE

SEQ ID NO: 15 - protein, 60 aa, Helicobacter pylori
GVPRILDLLDKYKIKITSHMSGRTVEMYPDRAKEIVQRGHEAAAHGWDWDNEFNMTAPQE

SEQ ID NO: 16 - protein, 44 aa, Helicobacter pylori
EDVLLKSVELIKDLTGKAPTGYVAPWWEFSNITNELLLKHGFKY

SEQ ID NO: 17 - protein, 44 aa, Helicobacter pylori
RDFIQRNVDIILKVTGQRAVGYNAPGLRGSVNILTVLNELGFVY

SEQ ID NO: 18 - protein, 60 aa, Helicobacter pylori
RLLKLFKKYHLPATWFSPGHSIETFSEQMKMIVDAGHEVGAHGYSHENPIAMTAKQEEDV

SEQ ID NO: 19 - protein, 60 aa, Helicobacter pylori
KILDVLKKHDVHATFFVTGHYLKTAPDLVKRMVKEGHIVGNHSWSHPDMTTISADKIKKE

SEQ ID NO: 20 - protein, 22 aa, Helicobacter pylori
LLKSVELIKDLTGKAPTGYVAP

SEQ ID NO: 21 - protein, 22 aa, Helicobacter pylori
LDAVSDKVKELTGQEGTVYVRP

SEQ ID NO: 22 - protein, 60 aa, Helicobacter pylori; identical to SEQ ID NO: 18

SEQ ID NO: 23 - protein, 60 aa, Helicobacter pylori; identical to SEQ ID NO: 19

SEQ ID NO: 24 - protein, 22 aa, Helicobacter pylori
LLKSVELIKDLTGKAPTGYVAP

SEQ ID NO: 25 - protein, 22 aa, Helicobacter pylori
LDAVSDKVKELTGQEGTVYVRP

SEQ ID NO: 26 - DNA, 26 bp, Helicobacter pylori
atcgcatgcaaaagaaatttagtggc

SEQ ID NO: 27 - DNA, 25 bp, Helicobacter pylori
atcaagctttttttctagggtttcg